(19) United States
(12) Patent Application Publication
(10) Pub. No.: US 2008/0183140 A1
(43) Pub. Date: Jul. 31, 2008

(54) SYRINGE CARTRIDGE SYSTEM

(75) Inventors: John Paproski, North Wales, PA (US); Paul Norton, Trumbauersville, PA (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: WEST PHARMACEUTICAL SERVICES, INC., Lionville, PA (US)

(21) Appl. No.: 11/947,509

(22) Filed: Nov. 29, 2007

Related U.S. Application Data
(60) Provisional application No. 60/867,685, filed on Nov. 29, 2006.

(51) Int. Cl. A61M 5/24; B65D 85/00; A61M 5/50

(52) U.S. Cl. 604/232; 206/571; 604/110

(57) ABSTRACT

The present embodiments provide for a syringe cartridge system that can be used in combination with multiple couplings and injection systems. The syringe cartridge system can be provided in a vacuum packaging assembly configuration. Alternatively, the syringe cartridge system can be provided with a coupler attached to a safety device, a hypodermic needle, or a Luer adaptor. The syringe cartridge system can also be used in combination with an automatic injector.

### CROSS REFERENCE TO RELATED APPLICATIONS

The present application claims the benefit of U.S. Provisional Patent Application No. 60/867,685 filed Nov. 29, 2006 entitled “Automatic Injection and Retraction Syringe Cartridge System,” the entire disclosure of which is hereby incorporated herein by reference.

### BACKGROUND OF THE INVENTION

Syringes are commercially packaged and shipped and often filled in containers that hold the syringes in an orderly fashion such that syringes can be automatically picked out of the shipping containers and placed onto an assembly line. Therefore, there is still a desire to directly sterilize, fill, and ship the syringes in a single container.

One dose syringes, such as staked needle syringes or syringes with a threadably attached needle, have the deficiency of allowing needle sticks and possible reuse of a contaminated needle. As such, there is an increased desire to prevent inadvertent needle sticks during the use and assembly of the syringes. In order to continue efficiently manufacturing, sterilizing, filling, and distributing the syringes, needle shields have been developed to attach to an end of needle syringes exposing the needle only during use.

Dispensers have also been developed for administering a single dose of medicine which have the general appearance of a pen or mechanical pencil. The dispensers, known as pen injectors or automatic injection and retraction syringes (“auto-injectors”) are typically large enough to hold a glass vial or cartridge of medicine, yet are small enough to fit conveniently in one's pocket or purse. Because the cartridges are shaped differently then a syringe, a different container and system is required for sterilization, filling, and shipment. It would therefore be advantageous to ship the cartridges directly from the filling container similar to the syringes above such that they are sterile and pyrogen free during and after their shipment. Such cartridges are not compatible with conventional syringes and needle shields.

As a result of the various systems known in the art for producing, shipping, and administering a single dose subcutaneous or intra muscular injection, it would be desirable to have a single system to manufacture and ship a universal medicine container or cartridge that is capable of being used directly as a syringe, used in conjunction with a syringe or needle shield, used in an auto-injector or any other syringe cartridge system.

### BRIEF SUMMARY OF THE INVENTION

The present invention provides for a syringe cartridge system comprising: a cartridge assembled to a needle hub assembly; and a packaging assembly that includes: a tray configured to support the cartridge, a nesting plate having a plurality of holes configured to receive the cartridge, the nesting plate positioned within the tray; and a flexible film defining an internal cavity configured to contain the tray, the nesting plate, and the cartridge.

The present invention also provides for a syringe cartridge system comprising: a cartridge that includes: a generally hollow tubular body having a distal and proximal end, a flange at the proximal end, and a septum at the distal end; and a needle hub assembly connectable to the cartridge, the needle hub assembly includes: a needle, a needle hub connected to the needle, a coupler operatively engaged with the needle hub and cartridge, and a guard surrounding the needle, needle hub, and coupler.

The present invention further provides for a syringe cartridge system comprising: a cartridge that includes: a generally hollow tubular body having a distal and proximal end, a flange at the proximal end, and a septum at the distal end; and a needle safety device connectable to the cartridge, the needle safety device includes: a needle, a needle hub connected to the needle and connectable to the cartridge, a slidable sleeve mounted to the needle hub, and a guard surrounding the needle, needle hub, and slidable sleeve.

The present invention also provides for a syringe cartridge injector comprising: a cartridge that includes: a hollow tubular body having a distal and proximal end, and a flange at the proximal end, a septum at the distal end, and a piston in the proximal end; and a syringe that includes: a hollow tubular body configured to receive the cartridge, a needle connected to the hollow tubular body, and a plunger connectable to cartridge.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a perspective view of a cartridge in accordance with an embodiment of the present invention;

FIG. 2 is an exploded view of a vacuum packaging assembly of a syringe cartridge system in accordance with an embodiment of the present invention;

FIG. 3a is a side view of a needle hub assembly assembled to the cartridge vial of FIG. 1;

FIG. 3b is a side cross-sectional view of the assembly of FIG. 3a;

FIG. 3c is a side view of the assembly of FIG. 3a showing the guard of the needle hub assembly removed and the syringe cartridge vial in a ready to use state;

FIG. 4 is an enlarged sectional view of a track mechanism on the interior surface of the guard of the embodiment in FIGS. 3a-3c;

FIG. 5 is a top perspective partial fragmentary view of the vacuum packaging assembly shown in FIG. 2 in assembled state;

FIG. 6a is a side view of a needle safety device assembled to the cartridge vial of FIG. 1 in accordance with another embodiment of the present invention;

FIG. 6b is a side cross-sectional view of the assembly of FIG. 6a;

FIG. 6c is a side the assembly of FIG. 6a showing the guard of the needle safety device removed and the syringe cartridge vial in a ready to use state;

FIG. 7 is a side perspective view of the needle safety device of FIGS. 6a-6c;

FIG. 8 is a partial side cross-sectional view of the needle safety device of FIGS. 6a-6c;

FIG. 9a is a side elevational view of the needle safety device shown in FIGS. 6a-6c in the storage position;

FIG. 9b is a cross-sectional view of the needle safety device shown in FIG. 9a taken along line B-B;

FIG. 9c is a side elevational view of the needle safety device shown in FIGS. 6a-6c in the armed position;

FIG. 9d is a cross sectional view of the needle safety device shown in FIG. 9c taken along line D-D;

FIG. 9e is a side elevational view of the needle safety device shown in FIGS. 6a-6c in the fully retracted position;

FIG. 9f is a cross-sectional view of the needle safety device shown in FIG. 9e taken along line F-F;

FIG. 9g is a side elevational view of the needle safety device shown in FIGS. 6a-6c in the fully extended position;

FIG. 9h is a cross-sectional view of the needle safety device shown in FIG. 9g taken along line H-H;

FIG. 10 is an elevational cross-sectional view of a cartridge needle safety syringe in accordance with another embodiment of the present invention;

FIG. 11 is a enlarged view of the cross-sectioned needle hub assembly of the embodiment in FIG. 10;

FIG. 12 is an enlarged elevational view of the needle hub assembly of the embodiment in FIG. 10;

FIG. 13 is an enlarged cross-sectional center perspective view of the needle safety device of the embodiment in FIG. 10 without the needle hub assembly present;

FIG. 14a is a side view of a Luer lock adapter assembled to the cartridge vial of FIG. 1 in accordance with another embodiment of the present invention;

FIG. 14b is a side cross-sectional view of the assembly of FIG. 14a;

FIG. 14c is a side view of the assembly of FIG. 14a showing the guard of the Luer lock adapter removed and assembled in a ready to use state with the cartridge; and

FIG. 15 is a perspective view of a cartridge based auto-injector in accordance with another embodiment of the present invention.

### DETAILED DESCRIPTION OF THE INVENTION

Certain terminology is used in the following description for convenience only and is not limiting. The words “right,” “left,” “upper,” and “lower” designate directions in the drawings to which reference is made. The terminology includes the words above specifically mentioned, derivatives thereof, and words of similar import.

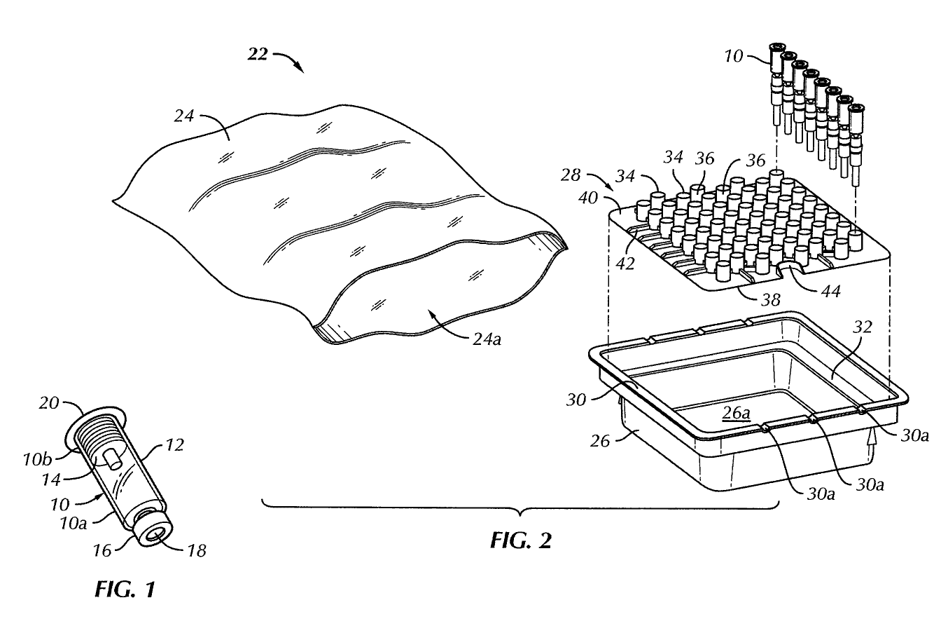

In an embodiment, the present invention provides for a syringe cartridge system (not numbered) that includes a cartridge 10 assembled to a needle hub assembly and a vacuum packaging assembly 22. Referring to FIG. 1, the cartridge 10 includes a vial body 12, a moveable piston 14, and an end cap 16. The vial body 12 (also referred herein as the cartridge vial) is generally constructed of glass, but may also be constructed of a high grade polymeric (i.e., plastic) material that is desirable in the marketplace because of the high resistance to breakage, low chemical extractables, and a reduced weight when compared to similar glass cartridges. The end cap 16 retains a stopper such as a pierceable septum 18 in the distal end of the vial body 12. The piston 14 can be configured for receiving and connecting to a conventional plunger (not shown) for administering a medicament within the vial body 12 in a manner well understood by those of ordinary skill in the art. The cartridge 10 has a distal end 10a for coupling with a needle assembly and a proximal end 10b, with the proximal end 10b having a flange 20.

Referring to FIG. 2, the syringe cartridge system also includes a vacuum packaging assembly 22. The vacuum packaging assembly 22 includes an air impervious flexible film shaped into a bag 24 or other container defining an internal cavity 24a, a tray 26, and a nesting plate 28. The vacuum packaging assembly 22 is similar to the Vacuum Packaging System disclosed in U.S. Patent Publication No. US 2006-0016156 A1 entitled “Vacuum Package System and Method,” the disclosure of which is hereby incorporated by reference in its entirety.

The tray 26 is preferably constructed out of a polymeric material. The tray 26 is not limited to polymeric materials and can be constructed out of any material sufficient for its intended use, such as a thermal foam plastic material or a machined material, as long as the tray 26 is able to take on the general size and shape and withstand the normal operating conditions of the tray 26. The tray 26 includes a mouth 30 and a lip 32 approximate the mouth 30. The mouth 30 preferably includes channels 30a at its peripheral edge to allow the vacuum bag 24 to draw air from the tray 26 when a vacuum is drawn on the internal cavity 24a of the bag 24. A completely flat mouth 30 could potentially cause the bag 24 to be breached or leave a pocket of air within the tray 26 when a vacuum is drawn from the internal cavity 24a exposing the tray 26 and cartridges 10 to external air.

The nesting plate 28 is configured with a plurality of holes 34. The holes 34 are preferably configured as cylindrical sleeves 36. Each cylindrical sleeve 36 releaseably receives one of the plurality of cartridges 10 and holds the cartridge 10 in a vertical orientation, when the nesting plate is assembled in the tray 26. When assembled with the tray 26, the peripheral edge 38 of the nesting plate 28 is positioned on the lip 32 of the tray 26 to support the nesting plate 28 within the tray 26. The nesting plate 28 is typically constructed out of a generally rigid, polymeric material. However, the nesting plate 28 can be constructed out of any material with sufficient rigidity to support the plurality of cartridges 10.

The nesting plate 28 is also configured with a generally planar base 40. The base 40 includes reinforcing ribs 42 extending generally perpendicular from the base 34. The reinforcing ribs 42 preferably extend to the peripheral edges 38 of the nesting plate 28 to provide stiffness and strength to the planner base 40. The nesting plate 28 can optionally include an arcuate shaped edge rib 44 that extends generally upwardly from the planar base 40 defining an edge hole. The edge hole allows a user to insert a finger or tool therethrough the edge hole to remove the nesting plate 28 from the tray 26. The edge rib 44 provides stiffness and strength for the base 40 approximate the edge hole. The edge hole and edge rib 44 are not limited to inclusion in the edge of the base 40 and can be positioned at nearly any location in the nesting plate 28. The cylindrical sleeves 36 which define the holes 34 through the nesting plate 28 are sized and shaped to accept the cartridges 10 and/or a cartridge assembly, such as a cartridge 10 assembled to a needle hub assembly. The nesting plate 28 is not limited to the inclusion of the reinforcing ribs 42 or cylindrical sleeves 36. For example, the nesting plate 28 can be constructed of a generally planar plate with holes formed therein for receipt of the cartridges 10. However, the cylindrical sleeves 36 and reinforcing ribs 42 are preferred for inclusion in the nesting plate 28 to better orient and space the cartridges 10 relative to the nesting plate 28 and to provide additional strength and stiffness for the nesting plate 28 when the assembly is inserted into the internal cavity 24a of the bag 24.

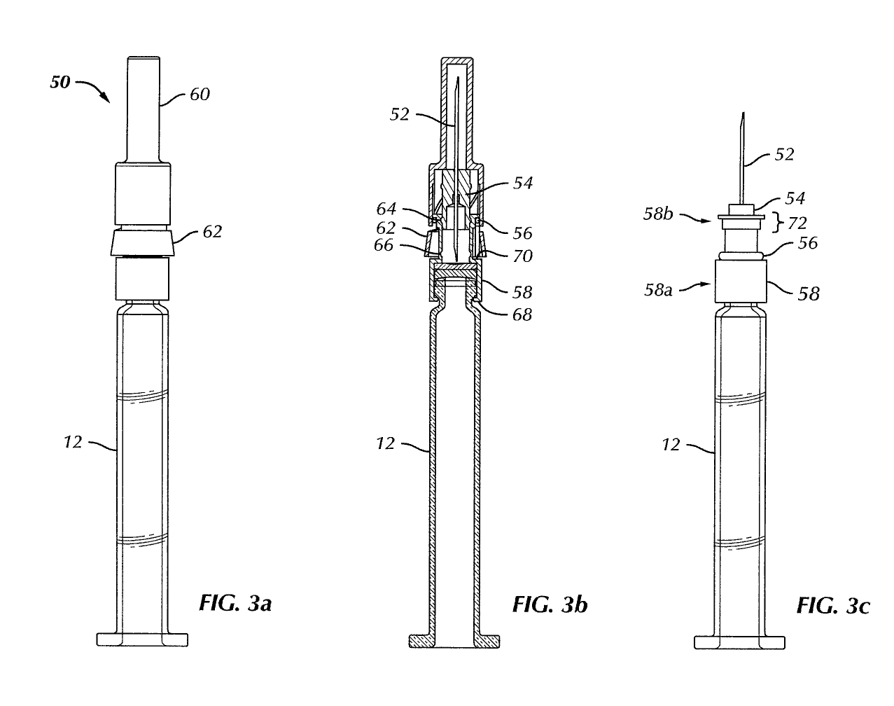

The syringe cartridge system also includes a staked needle assembly 50 as shown in FIGS. 3a-3c. The staked needle assembly 50 includes a needle 52, a needle hub 54, an optional sealing ring 56, a coupler 58, and guard 60 having a removable strip 62 such as a tear strip, a retaining strip, or a threaded retaining strip. The sealing ring 56, which can be fixed in position by the guard 60, advantageously facilitates maintaining the sterility of the staked needle assembly 50. In the present embodiment, the movable strip is configured as a tear strip 62. The needle 52 is permanently connected to the needle hub 54. The needle hub 54 is configured as a generally cylindrical tubular body having first 64 and second 66 snap-fits (such as detents) in cooperation with corresponding components on the coupler 58. For example, a male detent component can be positioned on the needle hub 54 and a female detent component can be positioned on the coupler 58. The first snap-fit 64 is positioned proximal the second snap-fit 66. In operation, the first 64 and second 66 snap-fits are used to controllably position the needle hub 54 from a first position, corresponding to the position of the first snap-fit 64 (as shown in FIG. 3b), to a second position corresponding to the position of the second snap-fit 66 (as shown in FIG. 3c).

The coupler 58 is configured as a generally hollow tubular body having a proximal end 58a and a distal end 58b. The proximal end 58a has a larger internal diameter than the internal diameter of the distal end 58b. The internal diameter of the proximal end 58a is configured to mate with the overall diameter of the cartridge 10 end cap 16. A lip 68 extends radially inwardly from the most proximal portion of the coupler 58 to allow for a snap-fit assembly of the coupler 58 to the cartridge 10 end cap 16. The top of the end cap 16 also abuts a ledge 70 defined by the distal end 58a. The distal end 58a of the coupler 58 has an inner opening in communication with the inner opening of the proximal end 58b, such that needle 52 can be freely passed through the coupler 58. The distal end 58b is also configured for sliding engagement with the needle hub 54. The distal end 58b also has a stepped flange 72 for receiving an optional sealing ring 56. The ledge 70 in concert with the stepped flange 72 is also configured to receive the width of the tear strip 62 so as to retain the guard 60, and if applicable the sealing ring 56, in a relatively fixed position when assembled to the coupler 58.

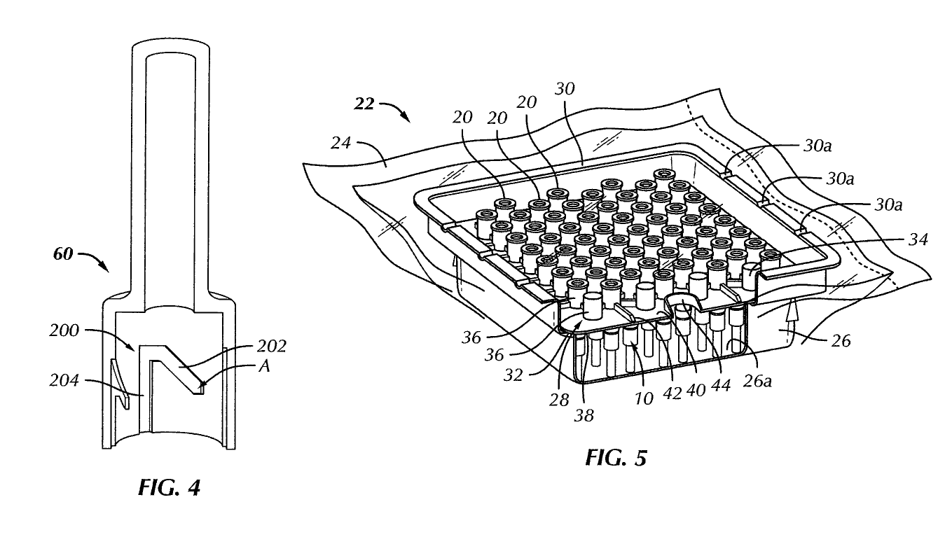

The guard 60 is configured with a tear strip 62 releasably attached to its proximal end. The guard 60 can also optionally be configured with a recessed track mechanism 200 (as shown in FIG. 4) configured on the internal surface of the guard 60 to facilitate the proximal movement of the needle hub 54 into the coupler 58. In the embodiment shown in FIG. 4, the guard 60 includes four tracks (only two tracks shown). The track mechanism 200 operates in conjunction with a series of lugs (not shown), preferably four lugs that are evenly spread apart and protrude radially outward from the distal end of the coupler 58. The lugs are initially retained in the track mechanism 200 at an initial position A. Additional, features such as an interference tab or bumps can be provided at the initial position A to relatively fixate the position of the lugs in the track mechanism 200. The track mechanism 200 is configured along the interior of the guard 60 and includes a circumferential track 202 and a vertical track 204. The circumferential track 202 preferably extends about 90° (i.e., a quarter turn) of the guard 60. The circumferential tracks 202 are configured to mate with the corresponding lugs (not shown) on the coupler 58 such that the lugs travel along the circumferential tracks 202 to corresponding vertical tracks 204. The vertical tracks 204 allow the lugs to travel in a vertical direction such that the guard 60 can be completely removed from the coupler 58.

The addition of the track mechanism 200 to the guard 60 inherently requires a user to push distally to disengage the guard 60 from the coupler 58 thereby automatically engaging the needle 52 to pierce the septum 18 and to fully seat the needle hub 54 within the coupler 58. This advantageously prohibits a user from accidentally forgetting to fully activate the cartridge syringe by not pushing the needle hub 54 proximally and fully seating the needle hub 54 before use.

Referring back to FIG. 3a, in an assembled state, the tear strip 62 is attached to the proximal end of the guard 60 and positioned in between the ledge 70 and stepped flange 72 of the coupler 58. In this initial position, the proximal end of needle 52 is positioned slightly above the end cap 16 so as not to pierce the septum 18.

FIG. 3a illustrates a cartridge vial 12 assembled to the needle hub assembly 50 (the cartridge vial 12 and needle hub assembly 50 hereinafter referred to as the cartridge staked needle) in an initial state. FIG. 3b illustrates a cross-sectional view of the needle hub assembly 50 assembled to the cartridge vial 12 in an initial state. FIG. 3c illustrates the needle hub assembly 50 assembled to the cartridge vial 12 after the tear strip 62 and guard 58 have been removed and the needle 52 positioned in a ready-to-use state.

To activate the cartridge staked needle, a user manually tears the tear strip 62 off the guard 60. Thereafter, the user manually depresses the guard 60 in the proximal direction either linearly or alternatively in a linear and twisting fashion as may be dictated by a track mechanism, to move the needle hub 54 from the first position to the second position, wherein the guard 60 bottoms out on the ledge 70. As the needle hub 54 is moved from the first position to the second position, the needle 52 is moved proximally to pierce the septum 18 to allow the flow of medicament from the cartridge 10 interior. Thereafter, the user can remove the guard 60 completely from the needle hub 54/coupler 58 assembly.

Referring back to FIG. 2, in assembling the syringe cartridge system, the nesting plate 28 is inserted into the tray 26 such that the peripheral edge 38 and the nesting plate 28 is positioned on the lip 32. The cartridge staked needles are then inserted into the holes 34 such that the flange 20 of the cartridges 10 are in facing engagement with the top of the cylindrical sleeve 36 and the distal end 10a is positioned proximate to the floor 26a. Such an orientation advantageously allows the cartridges 10 to be pre-filled and used in pre-filled syringe type applications. The general arrangement of cartridges 10 in the tray 26 is generally known to one having ordinary skill in the art using conventional syringes in place of the cartridges 10. The cartridges 10 and/or cartridge staked needles differ from conventional cartridges in that the cartridges 10 have the flange 20 on the proximal end 10b for supporting the cartridge 10 in the nesting plate 28 and for gripping the syringe in use. The array of cartridge staked needles generally have a common center to center distance such that robotic handling equipment is able to remove and insert these cartridges 10 from and into the nesting plate 28. A lid (not shown) can be engaged with the mouth 30 of the tray 26 to close the mouth 30, however, the lid is not necessary for the operation of the vacuum packaging system 22. Alternatively, cartridges 10 alone can be assembled in the tray 26 without being assembled to the needle hub assembly 50. The tray 26 is positioned into the internal cavity 24a of the vacuum bag 24 and the internal cavity 24a is purged and evacuated to a pre-determined pressure, such as below atmosphere pressure.

The syringe cartridge system can be pre-filled with a medicament or a plurality of medicaments prior to the bag 24 being sealed or at any time prior to the system be processed through a sterilization process. Alternatively, the syringe cartridge system can be packaged in the vacuum packaging assembly without pre-filling the cartridges 10.

The assembled configuration of the syringe cartridge system is shown in FIG. 5. The lip 32 is provided to vertically support the nesting plate 28 above the floor 26a of the tray 26 and to optionally space the distal end of the cartridge staked needles from the floor 26a. The nesting plate 28 can optionally be mechanically fastened, clipped, bonded or otherwise mounted to the tray 26. In addition, the nesting plate 28 can include legs (not shown) to support the nesting plate 28 above the floor 26a.

The present packaging assembly advantageously addresses the problem of packaging effects associated with non-permeable packaging as non-permeable packaging is subject to the effects of changing atmospheric pressure. Part of the package function is to contain the cartridge staked needles in the nesting plate 28 within the tray 26 to reduce movement of the cartridge staked needles and maintain the cartridge staked needles in a sterile, pyrogen free, and particulate matter free condition until they are ready to be filled with the medical products. If a non-permeable package expands due to reduced atmospheric pressure encountered primarily during air shipment or alternate high elevation shipment, the package will no longer hold the cartridge staked needles in the position in the nesting plate 28, potentially resulting in rubbing of the cartridge staked needles against the nesting plate 28 or bumping into each other. A secondary effect is that the package may be stressed at weld points or other stress risers and rupture during shipping. A rupture of the package could cause a leak and the sterility of the contents may likely be compromised.

The present embodiment addresses this packaging problem by providing a vacuum package using a flexible film vacuum bag 24. A multiple-layer film comprised of at least one layer with very low gas permeability is configured into the vacuum bag 24 and sized to cover or contain the tray 26. The tray 26 containing the cartridge staked needles mounted in the nesting plate 28 is placed into the vacuum bag 24 and the bag 24 is evacuated and sealed. With the air removed, the bag 24 clings tightly to the tray 26 and the proximal end 10b or flanges 20 of the cartridge staked needles holding them firmly in the nesting plate 28. The negative effects of reduced atmospheric pressure are counteracted because the bag 24 generally will not loosen its grip on the cartridge staked needles until the seal of the bag 24 is broken. For this reason, it is desirable to reach a level of vacuum inside the bag 24 at least equal to or nearly equal to the pressure encountered in aircraft shipment which is typically eight inches of mercury (8 in. Hg) below standard atmospheric pressure or equivalent to eight thousand feet (8,000 ft.) above sea level. Held tightly in the nesting plate 28, the cartridge staked needles are less likely to be scratched by contact with the nesting plate 28 and other packaging materials. The vacuum in the bag 24 also serves as a ready indicator of package integrity since even the slightest leak or breach will cause the bag 24 to relax, which will be visually apparent (i.e., a visual indicator) to an operator inspecting the bag 24. A bag 24 with a leak would be thus readily identified visually as having been breached. Further, maintenance of the vacuum pressure in the bag 24 indicates that the tray 26 and its external surfaces have been maintained in their sterile, pyrogen free, and particulate matter free condition. Even without the bag 24, the tray 26 allows convenient transfer of the cartridge staked needles to an applicator as described in detail below.

In another embodiment, the present invention provides for a syringe cartridge system that includes a cartridge vial 12 (as shown in FIG. 1) and a needle hub assembly 50 (as shown in FIGS. 3a-3c) as previously described above. The cartridge vial 12 can be provided in a vacuum package assembly while the needle hub assembly 50 provided separately. Thus, the cartridge vial 12 can be assembled to the needle hub assembly 50 at the time of use or point of care.

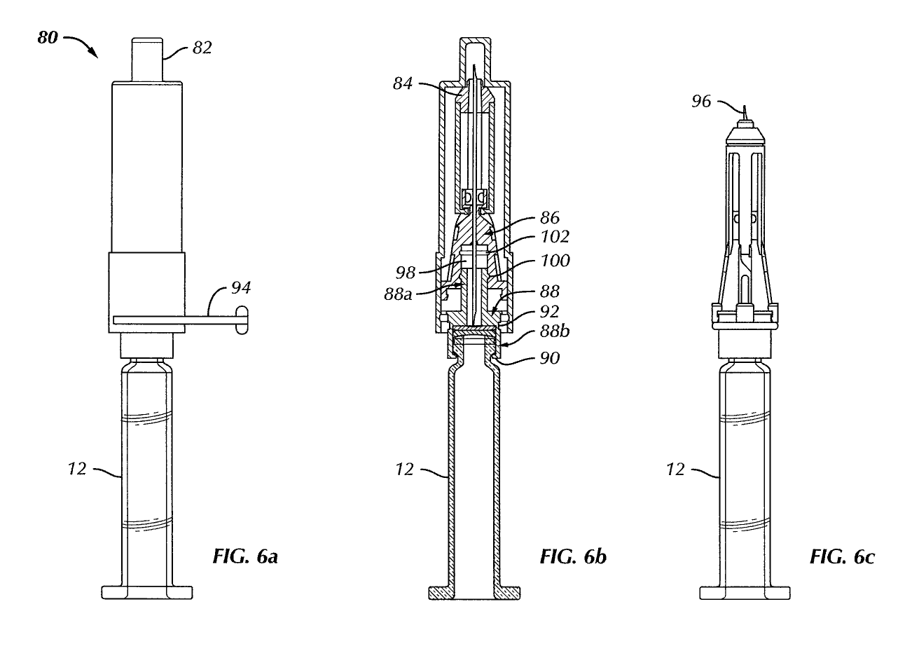

In a further embodiment, the present invention provides for a syringe cartridge system that includes a cartridge vial 12 (as shown in FIG. 1) and a needle safety device 80 (as shown in FIGS. 6a-6c), the assembly hereinafter referred to as the cartridge needle safety syringe I. The needle safety device 80 is an integrated safety device compatible with the cartridge vial 12 and similar to the needle safety device generally described in published European Patent Application Number 1 535 640 A1 ('640 Application) and in U.S. patent application Ser. No. 11/815,475 ('475 Application), the disclosures of which are hereby incorporated by reference in their entirety.

As shown in FIGS. 6a-6b, the needle safety device 80 includes a guard 82, a slidable sleeve 84, a needle hub 86, and a coupler 88. The coupler 88 is a generally hollow tubular body with a distal end 88a and a proximal end 88b. The distal end 88a has a smaller overall inner and outer diameter than the proximal end 88b. The internal diameter of the proximal end 88b is configured to mate with the overall diameter of the cartridge 10 end cap 16. A lip 90 extends radially inwardly from the most proximal portion of the coupler 88 to allow for a snap-fit assembly of the coupler 88 to the cartridge 10 end cap 16. A circumferential groove 92 is also configured on the proximal end 88b of the coupler 88. The circumferential groove 92 operates with a removable strip such as a retaining wire 94, which in combination with the circumferential groove 92, functions to retain the guard 82 on the coupler 88 in a relatively fixed position. The distal end 88a of the coupler 88 has an inner opening in communication with the inner opening of the proximal end 88b, such that the needle 96 can be freely passed through the coupler 88. The distal end 88a is also configured to operatively engage the needle hub 86.

The needle hub 86 has a generally conical shape tapering down towards its distal end and a needle 96 permanently affixed thereto. At its most proximal end, the needle hub 86 is configured to seat on the proximal end 88b of the coupler 88. The main body of the needle hub 86 has a generally hollow cylindrical interior 98 configured to mate with the distal end 88a of the coupler 88 such that the needle hub 86 can be fully seated on the proximal end 88b of the coupler 88. A pair of detents 100, 102 configured on the needle hub 86 and coupler 88 control the seating positions of the needle hub 86 on the coupler 88. In operation, the needle hub 86 is moved from a first position (as shown in FIG. 6b), when the needle hub 86 is secured to the coupler 88 by a first detent 100 located proximally from the second detent 102, to a second position (as shown in FIG. 6c), when the needle hub 86 is secured to the coupler 88 by the second detent 102. The second position corresponds to the needle hub 86 being fully seated on the coupler 88.

The needle 96 is positioned on the needle hub 86 such that when in the first position, the proximal end of the needle 96 is slightly above the end cap 16. However, when the needle hub 86 is moved to the second position, the proximal end of the needle 96 pierces the septum 18 to allow fluid from within the cartridge to pass through the needle 96.

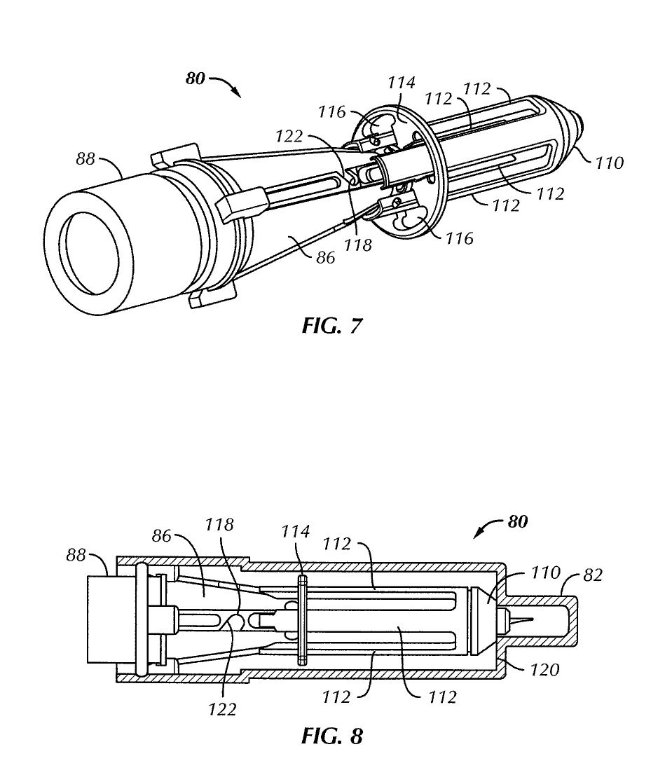

As shown in FIG. 7, the slidable sleeve 110 is engaged with the needle hub 86 by cantilever arms 112 having knobs (not shown) extending radially inwardly from its proximal end. The needle safety device 80 also includes a circular retaining ring 114, disposed around the cantilever arms 112 of the slidable sleeve 110. The retaining ring 114 is engaged with the distal end of the needle hub 86 and functions to retain the slidable sleeve 110 on the needle hub 86. The retaining ring 114 includes four evenly spaced apart holes 116 for accommodating each of the four cantilever arms 112 of the slidable sleeve 110 with sufficient clearance to allow sliding and flexing of the cantilever arms 112 when captured within the retaining ring 114. The flexing of the cantilever arms 112 creates a leaf spring arrangement of the sliding sleeve 110.

As shown in FIG. 8, a guard 82 is removably mounted on the coupler 88 such that it covers the slidable sleeve 82 and needle hub 86 assembly. The guard 82 can also optionally be configured with a track mechanism (not shown) configured on the internal surface of the guard 82 to facilitate the proximal movement of the needle hub 86 into the coupler 88, as previously described in the above embodiments.

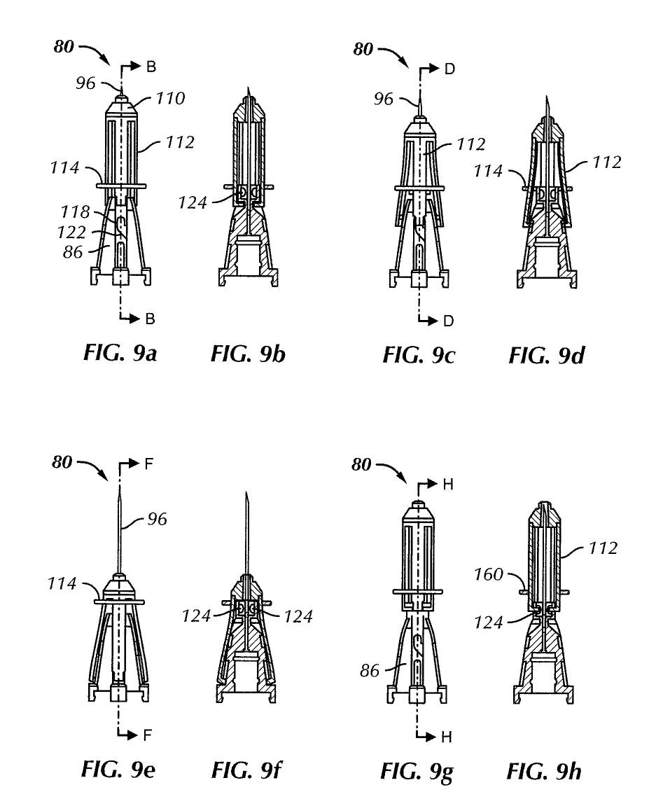

As shown in FIGS. 9a and 9b there is little load on the cantilever arms 112 initially, but the load is sufficient to hold the slidable sleeve 110 to the needle hub 86. The distal end of slidable sleeve 110 abuts the end of the guard (as shown in FIG. 8). To set the needle safety device 80 in the ready or loading position as shown in FIGS. 9c and 9d, the needle hub 86 can be urged into the guard 82 forcing the cantilever arms 112 up into the needle hub 86 until at least one of the cantilever arms 112 snap over a catch 118. The catch 118 is a groove in the needle hub 86 that prevents retraction of the slidable sleeve 112 along the same path in which the slidable sleeve 112 entered the catch 118. The needle hub 86 is prevented form further displacement by a shoulder 120 in a guard 82. The needle safety device 80 can also be set in the ready or loading position as shown in FIGS. 9c and 9d without the use of the guard 82 as further described below.

The needle 96 is initially partially exposed when the needle safety device 80 is in the storage position as shown in FIGS. 9a and 9b. The guard 82 covers the needle 96 until the needle safety device 80 is administered. When a shot is to be administered, the needle safety device 80 is either in the storage position as shown in FIGS. 9a and 9b or the loading or ready to use position as shown in FIGS. 9c and 9d. The guard 82 is removed and the exposed needle 96 is inserted into the skin (not shown) until the slidable sleeve 110 comes into contact with the skin. Further movement of the needle 96 into the skin urges the slidable sleeve 110 up the needle hub 86 and thus the cantilever arms 112 are forced further outwardly by the conical surface of the needle hub 86. The slidable sleeve 110 is urged into the catch 118 and then forced against a cam surface 122 (as shown in FIG. 8) which causes the slidable sleeve 110 and cantilever arms 112 to rotate with respect to the needle hub 86. As the slidable sleeve 110 is urged further up the needle hub 86, more of the needle 96 is exposed and inserted deeper into the skin. When the needle 96 is withdrawn from the skin, the resulting spring leaf force of the cantilever arms 112 causes the slidable sleeve 110 to rapidly extend into a protective position shown in FIGS. 9g and 9h covering the tip of the needle 96 and thereby inhibiting accidental needle sticks 96 thereafter. The slidable sleeve 110 bypasses the catch 118 because the cantilever arms 112 are now radially offset from the catch 118 and are unimpeded as they slide down the needle hub 86. In the protective position, the knobs of the cantilever arms 112 are locked into holes 124 near the distal most end of the needle hub 86 such that the slidable sleeve 112 is no longer slidable with respect to the needle hub 86 and the needle 96 remains completely covered to prevent accidental sticks. The entire assembly including the cartridge 10, the coupler 88, and the needle safety device 80 can then be safely disposed. Alternatively, the cartridge 10 and or the coupler 88 can be removed from the needle safety device 80 and recycled or reused, disposing only the needle safety device 80.

In sum, the needle safety device 70 includes a needle 96, a needle hub 86 surrounding the needle 96, a slidable sleeve 110 slidably mounted on the needle hub 86 and a guard 82 surrounding the needle 96, needle hub 86, and slidable sleeve 110. The needle hub 86, slidable sleeve 110, and guard 82 have a receiving end which is proximal to the tip of the needle 96 and an injection end which is distal to the tip of the needle 96. The receiving end of the needle hub 86 is suitable for connection to an injection device such as a syringe cartridge system. The slidable sleeve 110 is adapted to slide in the direction of the length of the needle 96 between an extended position in which the injection end of the needle 96 is located inside the slidable sleeve 110 and a retracted position in which the injection end of the needle 96 projects from the slidable sleeve 110, via an intermediate position between the extended position and the retracted position in which the injection end of the needle 96 projects partially from the slidable sleeve 110, such that, in use, the slidable sleeve 110 is moved into the intermediate position for injection into a patient. Then as the needle 96 is inserted into a patient, the slidable sleeve 110 is caused to move into the retracted position, and in moving into the retracted position a resultant force is generated such that on removal of the needle 96 from the patient, the resultant force causes the slidable sleeve 110 to move towards the injection end of the needle hub 86 and into the extended position. The needle safety device further includes a locking mechanism capable of retaining the slidable sleeve 110 in the extended position after removal of the needle from the patient. The guard 82 is also releasably mounted on the needle hub 86 and slidable sleeve 110 such that the injection end of the guard 82 covers at least the injection end of needle 96 and the receiving end of the guard 82 has an open portion to expose the receiving end of the needle hub 86, and by causing the guard 82 to be moved in a direction towards the receiving end of the needle hub 86, the guard 82 engages with the slidable sleeve 110 which is retracted from the extended position to the intermediate position.

The cartridge needle safety syringe I can optionally be configured with a track mechanism as previously described in the above embodiment. Moreover, the overall dimensions of the cartridge needle safety syringe I can advantageously be configured and sized to fit within the vacuum packaging assembly as described in the above embodiment.

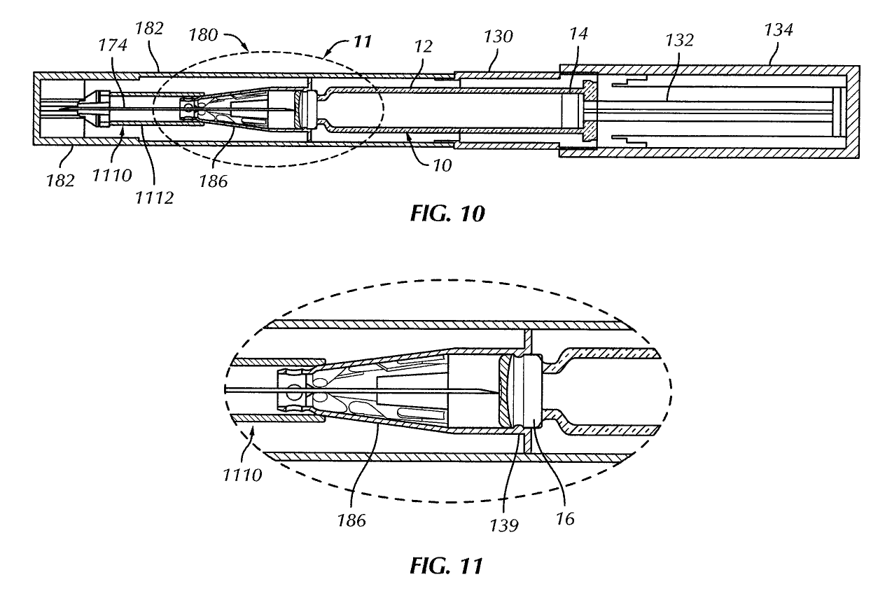

FIG. 10 illustrates another embodiment of the syringe cartridge system having a cartridge (similar to that as shown in FIG. 1) and a needle safety device 180 (similar to that as shown in FIGS. 6a-6c and described in the above cartridge needle safety syringe I embodiment), the assembly hereinafter referred to as the cartridge needle safety syringe II. The cartridge needle safety syringe II includes a needle safety device 180 having a slidable shield 1110 with cantilever arms 1112, and a needle hub 186. The cartridge needle safety syringe II also includes a cartridge 10, a holder 182, a housing 130, a plunger 132, and a cap 134.

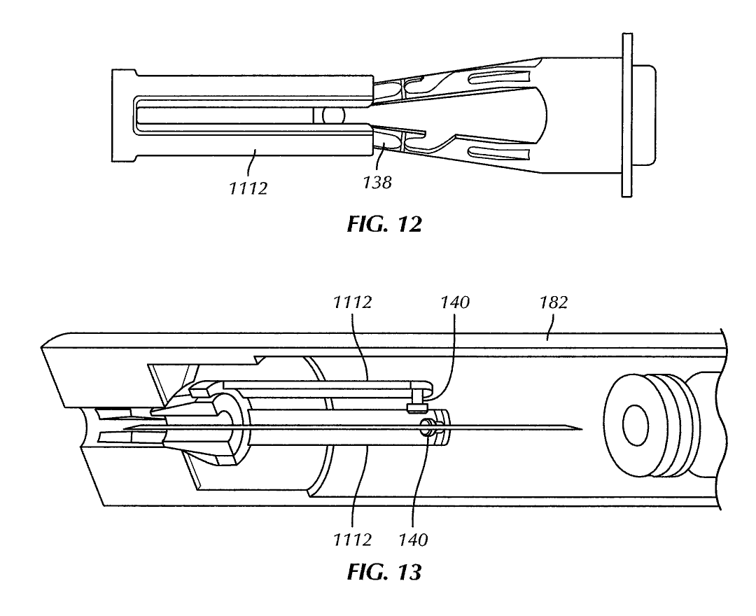

As shown in FIGS. 11 and 12, the slidable shield 1110 is connected to the needle hub 186 via cantilever arms 1112 in a manner similar to that as described for the cartridge needle safety syringe I embodiment. However, the needle safety device 180 of the present embodiment does not include a retaining ring. The cantilever arms 1112 are attached to the needle hub 186 by knobs 140 (as shown in FIG. 13) extending radially inwardly from the cantilever arms 1112 and engaging corresponding notches 138. All other functions and operations of the needle safety device 180 retraction and activation are as disclosed for the cartridge needle safety syringe I embodiment described above.

The present embodiment also differs from the cartridge needle safety syringe I embodiment in that the needle hub 186 is directly connected to the cartridge 10 end cap 16 as best shown in FIG. 11. The needle hub 186 is connected to the cartridge 10 by a snap-fit assembly such as, for example a detent 139. The male component of the detent 139 can be positioned on the proximal end of the needle hub 186 and the corresponding female component of the detent 139 can be positioned on the end cap 16.

The overall assembly of the cartridge needle safety syringe II is housed within the holder 182, housing 130, and cap 134 assembly as shown in FIG. 10. The housing 130 is secured to the cap 134 by a first threaded connection such as right-handed threads (not shown) while the housing 130 is secured to the holder 182 by a second threaded connection such as left-hand threads (not shown). The threads on the cap 134 are also configured to be operatively engaged with the proximal end of the cartridge 10 such that as the threads on the cap 134 move distally, the threads apply a distal force to the proximal end of the cartridge 10 such that the cartridge 10 moves distally. For example, the threads can be engaged with the cartridge 10 by a lip, an under or oversized thread, a stylus, or any other means such that as the cap 134 is moved distally, the cartridge 10 is also forced to move distally relative to the housing 130. In the assembled state, the right-hand threads of the holder 182 and housing 130 are fully engaged, whereas the left-hand threads are not. The overall dimensions of the cartridge needle safety syringe II can also advantageously be configured and sized to fit within the vacuum packaging assembly as described in the above embodiment.

To activate the cartridge needle safety syringe II, a user can grasp the cap 134 and holder 182 and rotates the cap 134 in a clockwise direction such that the right-hand threads of the cap 134 engage the corresponding threads on the housing 130. As the cap 134 is threaded, the threads of the cap 134 push the cartridge 10 distally within the holder 182 such that the male component of the detent 139 locks or snaps over the proximal end of the end cap 16 and the needle pierces through the septum 18. The holder 182 can also optionally be configured such that the distal movement of the cartridge 10 also sets the slidable shield 1110 into the ready to use position on the needle hub 186.

The point at which the needle hub 186 is fully set on the cartridge 10, represents the point at which the right-hand threads are fully engaged such that further clockwise rotation of the cap 134 starts to unscrew the initially fully engaged left-hand threads holding the housing 130 and holder 182 together. The user continues unscrewing the left-hand threads until the housing 130 and cap 134 assembly can be removed allowing the user to remove the syringe cartridge from the holder 182. In sum, as the user starts a clockwise rotation of the cap 134, it operates to engage the first threaded connection and subsequently to disengage the second threaded connection.

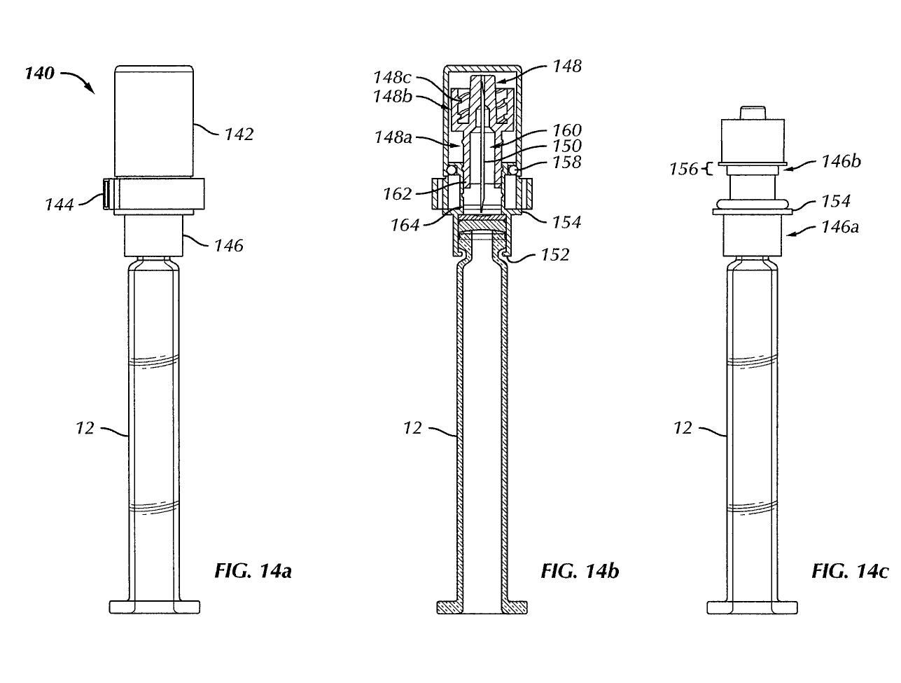

In yet another embodiment, the present invention provides for a syringe cartridge system that includes a cartridge vial 12 (as shown in FIG. 1) and Luer lock adapter 140 (the assembly hereinafter referred to as the cartridge Luer lock) as shown in FIGS. 14a-14c. Luer lock adapter 140 includes a guard 142 having a removable strip such as a tear strip 144, a coupler 146, a needle hub 148, and a needle 150 permanently attached to the needle hub 148.

The coupler 146 has a generally hollow tubular body with a proximal end 146a and a distal end 146b. The distal end 146b has a smaller overall inner and outer diameter than the proximal end 146a. The internal diameter of the proximal end 146a is configured to mate with the overall diameter of the cartridge 10 end cap 16. A lip 152 extends radially inwardly from the most proximal portion of the coupler 146 to allow for a snap-fit assembly of the coupler 146 to the cartridge 10 end cap 16. The top of the end cap 16 also abuts against a ledge 154 defined by the distal end 146b. The ledge 154 also extends radially outward beyond the outer diameter of the proximal end 146a to retain the tear strip 144. The distal end 146b of the coupler 146 has an inner opening in communication with the inner opening of the proximal end 146a, such that the needle 150 can be freely passed through the coupler 146. The distal end 146b has an inner diameter configured to mate with and receive the needle hub 148. The distal end 146b also has a stepped flange 156 for receiving an optional sealing ring 158. The ledge 154 in concert with the stepped flange 156 is configured to receive the width of the tear strip 144 and retain the guard 142 in a fixed position when assembled to the coupler 146.

The needle hub 148 has a proximal end 148a and a distal end 148b. The distal end 148b is configured with Luer lock threads 148c which are readily known in the art. The proximal end 148a is of a generally cylindrical configuration having a hollow interior 160. The proximal end 148a is configured to slidably engage with the distal end 146b of the coupler 146.

A pair of snap-fits 162 and 164 is configured on the needle hub 148 and coupler 146 such that the needle hub 148 can be moved from a first position (as shown in FIG. 14b) to a second position (as shown in FIG. 14c) similar in operation to that of the needle safety device embodiment above. It is to be understood that the present embodiment is not limited to the use of snap-fits, but intended to cover any means capable of relatively fixating the needle hub 148 in a first position and a second position within the coupler 146, such as with detents, interference fits, and the like.

The guard 142 is configured with a tear strip 144 releasably attached to its proximal end. The guard 142 can also optionally be configured with a track mechanism (not shown) configured on the internal surface of the guard 142 to facilitate the proximal movement of the needle hub 148 into the coupler 146, as previously described in the above embodiments.

In an assembled state, the tear strip 144 is attached to the proximal end of the guard 142 and positioned in between the ledge 154 and stepped flange 156 of the coupler 146. In this initial position, the proximal end of the needle 150 is positioned slightly above the end cap 16 so as not to pierce the septum 18.

To activate the cartridge Luer lock, a user can manually tear the tear strip 144 off the guard 142. Thereafter, the user depresses the guard 142 in the proximal direction either linearly or alternatively in a linear and twisting fashion as may be dictated by a track mechanism, to move the needle hub 148 from the first position to the second position. As the needle hub 148 is moved from the first position to the second position, the needle 150 is moved proximally to pierce the septum 18 to allow the flow of medicament from the cartridge 10 interior. Thereafter, the user can remove the guard 142 completely from the needle hub 148/coupler 146 assembly to further attach a corresponding needle or device to the needle hub 148.

In yet a further embodiment, the present invention provides for a syringe cartridge injector. The syringe cartridge injector includes a cartridge 10 (as shown in FIG. 1) and a syringe assembly. The syringe assembly can be any syringe assembly configured to receive the cartridge 10 so that the syringe assembly and cartridge 10 can function substantially as a conventional single use syringe. Preferably, the syringe assembly is an automatic injector (also known as an auto-injector) similar to those further described in U.S. Patent Application Publication No. 2006/0178631 and U.S. Pat. No. 6,387,078, the disclosures of which are hereby incorporated by reference in their entirety.

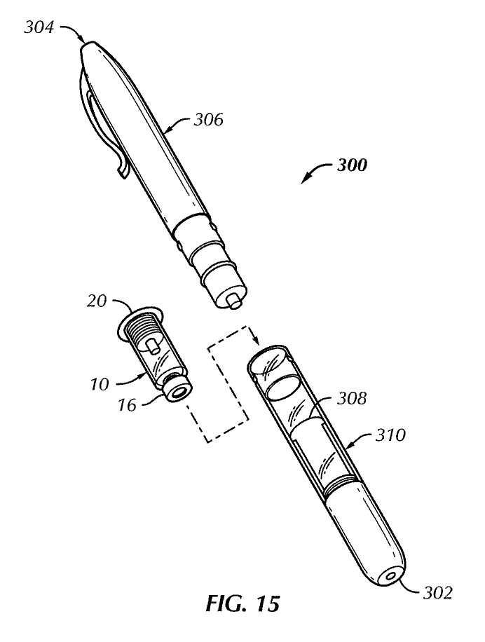

As shown in FIG. 15, the auto-injector 300 extends a hypodermic needle (not shown) from within the auto-injector 300, injects a single pre-measured dose of medicine from the cartridge 10 or the entire contents of the cartridge 10 into a user, and automatically retracts the hypodermic needle into the assembly after the injection is completed. The auto-injector 300 defines an injection end 302 for placement against a user where a needle extends and an activation end 304 for activating the injection assembly 306. The auto-injector 300 can include an edge 308 defining the start of a smaller diameter of the retraction assembly 310 for engaging with the flange 20 of the cartridge 10. The flange 20 may alternatively be sized and configured to fit into conventional syringes without an edge 308. In this configuration, the flange 20 would abut the inside of the retraction assembly 310 and the body of the cartridge 10 would be spaced from the auto-injector 300. Further details and operation of exemplary auto-injectors is further described in U.S. Patent Application Publication No. 2006/0178631 and U.S. Pat. No. 6,387,078. Although the present embodiment has been described with reference to an auto-injector, it is within the scope and intent that the present embodiment can be used with any syringe capable of receiving the cartridge 10.

From the foregoing description, it can be seen that the present invention provides for a novel and versatile syringe cartridge system capable of being used with multiple systems. For example, the cartridge can be configured in combination with a vacuum packaging assembly, needle hub assembly, needle safety device, Luer lock assembly, or an auto-injector. One of the main benefits of such a syringe cartridge system is that existing and conventional filling, dispensing systems, and packaging systems can be utilized without having to be modified.

It will be appreciated by those skilled in the art that changes could be made to the embodiments described above without departing from the broad inventive concept thereof. For example, additional components and steps can be added to the various syringe cartridge systems. It is to be understood, therefore, that this invention is not limited to the particular embodiment disclosed, but it is intended to cover modifications within the spirit and scope of the present invention as defined by the appended claims.

We claim:

1. A syringe cartridge system comprising:
a cartridge assembled to a needle hub assembly; and
a packaging assembly that includes:
a tray configured to support the cartridge;
a nesting plate having a plurality of holes configured to receive the cartridge, the nesting plate positioned within the tray; and
a flexible film defining an internal cavity configured to contain the tray, the nesting plate, and the cartridge.

2. The syringe cartridge system of claim 1, wherein the cartridge comprises:
a generally hollow tubular body having a distal and proximal end;
a flange at the proximal end; and
a septum at the distal end.

3. The syringe cartridge system of claim 1, wherein the packaging assembly is a vacuum packaging assembly and the internal cavity is evacuated to a predetermined pressure level.

4. The syringe cartridge system of claim 1, wherein the needle hub assembly comprises:
a needle;
a needle hub connected to the needle;
a coupler operatively engaged with the needle hub and cartridge; and
a guard surrounding the needle, needle hub, and coupler.

5. The syringe cartridge system of claim 4, wherein the needle hub assembly further comprises a slidable sleeve mounted to the needle hub.

6. The syringe cartridge system of claim 4, wherein the needle hub assembly further comprises Luer lock threads.

7. The syringe cartridge system of claim 4, wherein the guard includes a removable retaining strip.

8. The syringe cartridge system of claim 4, wherein the guard comprises:
a generally hollow tubular body having a distal and proximal end;
a removable strip connected to the proximal end; and
a track mechanism configured on an internal surface of the generally hollow tubular body.

9. The syringe cartridge system of claim 8, wherein the track mechanism comprises:
a circumferential track; and
a vertical track connected to the circumferential track.

10. The syringe cartridge system of claim 8, wherein the track mechanism is configured to operatively engage a lug on the coupler.

11. The syringe cartridge system of claim 1, wherein the flexible film is sealed defining a sealed internal cavity and the cartridge pre-filled prior to the flexible film being sealed.

12. A syringe cartridge system comprising:
a cartridge that includes:
a generally hollow tubular body having a distal and proximal end;
a flange at the proximal end; and
a septum at the distal end; and
a needle hub assembly connectable to the cartridge, the needle hub assembly includes:
a needle;
a needle hub connected to the needle;
a coupler operatively engaged with the needle hub and cartridge; and
a guard surrounding the needle, needle hub, and coupler.

13. The syringe cartridge system of claim 12, wherein the needle hub includes Luer lock threads at a distal end of the needle hub.

14. The syringe cartridge system of claim 12, wherein the guard includes a removable strip configured to retain the guard on the coupler.

15. The syringe cartridge system of claim 12, wherein the guard comprises:
a generally hollow tubular body having a distal and proximal end;
a removable strip connected to the proximal end; and
a track mechanism configured on an internal surface of the generally hollow tubular body.

16. The syringe cartridge system of claim 15, wherein the track mechanism comprises:
a circumferential track; and
a vertical track connected to the circumferential track.

17. The syringe cartridge system of claim 15, wherein the track mechanism is configured to operatively engage a lug on the coupler.

18. A syringe cartridge system comprising:
a cartridge that includes:
a generally hollow tubular body having a distal and proximal end;
a flange at the proximal end; and
a septum at the distal end; and
a needle safety device connectable to the cartridge, the needle safety device includes:
a needle;
a needle hub connected to the needle and connectable to the cartridge;
a slidable sleeve mounted to the needle hub; and
a guard surrounding the needle, needle hub, and slidable sleeve.

19. The syringe cartridge system of claim 18, wherein the needle safety device further includes a coupler operatively engaged with the needle hub configured to connect the needle hub to the cartridge.

20. The syringe cartridge system of claim 18, wherein the guard includes a removable strip configured to retain the guard on the coupler.

21. The syringe cartridge system of claim 18, wherein the guard comprises:
a generally hollow tubular body having a distal and proximal end;
a removable strip connected to the proximal end; and
a track mechanism configured on an internal surface of the generally hollow tubular body.

22. The syringe cartridge system of claim 21, wherein the track mechanism comprises:
a circumferential track; and
a vertical track connected to the circumferential track.

23. The syringe cartridge system of claim 21, wherein the track mechanism is configured to operatively engage a lug on the coupler.

24. The syringe cartridge system of claim 18, further comprising:
a holder configured to house the distal end of the needle safety device;
a cap configured to house the proximal end of the cartridge; and
a housing that includes:
a first threaded connection, operatively engaged with the proximal end of the cartridge, configured to connect with the cap; and
a second threaded connection configured to connect with the holder;
wherein rotation of the cap in a first direction operates to engage the first threaded connection and disengage the second threaded connection.

25. The syringe cartridge system of claim 24, wherein rotation of the cap in the first direction operatively engages the first threaded connection such that the first threaded connection drives the cartridge in the distal direction.

26. A syringe cartridge injector comprising:
a cartridge that includes:
a hollow tubular body having a distal and proximal end; and
a flange at the proximal end;
a septum at the distal end; and
a piston in the proximal end; and
a syringe that includes:
a hollow tubular body configured to receive the cartridge;
a needle connected to the hollow tubular body; and
a plunger connectable to cartridge.

27. The syringe cartridge injector of claim 26, wherein the cartridge is pre-filled with a medicament prior to assembly with the syringe

28. The syringe cartridge injector of claim 26, wherein the syringe is an automatic injector.

29. The syringe cartridge injector of claim 28, wherein the automatic injector comprises:
an injection assembly; and
a retraction assembly, wherein the retraction assembly is configured to receive the cartridge.

30. The syringe cartridge injector of claim 29, wherein the retraction assembly includes an edge for retaining the cartridge.
